(19) United States
(12) Patent Application Publication
(10) Pub. No.: US 2006/0257479 A1
(43) Pub. Date: Nov. 16, 2006

(54) Long-acting molecules in sustained release formulations

(75) Inventors: Simon Bjerregaard Jensen, Frederiksberg (DK); Lars Fogh Iversen, Holte (DK); Christian Rischel, Copenhagen S (DK); Mats Reslow, Lund (SE)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/395,770

(22) Filed: Mar. 31, 2006

Related U.S. Application Data
(63) Continuation of application No. PCT/DK04/00684, filed on Oct. 8, 2004.

(30) Foreign Application Priority Data
Oct. 10, 2003 (DK) PA 2003 01496

(51) Int. Cl. A61K 9/22; A61K 38/27

(52) U.S. Cl. 424/468; 514/12

(57) ABSTRACT

Sustained release formulations comprising molecules modified so as to have a reduced clearance is provided.

### FIELD OF THE INVENTION

The present invention relates to sustained release formulation comprising molecules that have been modified to obtain a reduced plasma clearance.

### BACKGROUND OF THE INVENTION

A large number of proteins including hormones, cytokines, antibodies are used as therapeutic agents. Due to the progress in biopharmaceutical technology and the market success the number will increase dramatically the forthcoming years.

Oral administration of protein drugs is generally not possible due to the proteolytic activity in the gastro intestinal tract and/or an insufficient absorption due to the large size of the these macromolecules. Currently the only feasible administration route for proteins is the parenteral route with the related pain and inconvenience for the patient.

Most protein and peptide drugs have a relatively short plasma half-life, which means that they are cleared fast from the systemic circulation.

A sustained release formulation is a formulation capable of releasing a drug, e.g. into the plasma, after administration in a controlled manner. Typically, the drug may be released over hours, days, weeks or even months.

Once a drug is released from a sustained release formulation into the systemic circulation, it is cleared from the plasma with the same rate as if it had been injected in a non-sustained release formulation. It therefore follows from the extended period of time over which a sustained release formulated drug exerts its activity, that the drug load in a sustained release formulation has to be large. The loading is often limited in sustained release formulations to about 5-15%. Administering higher doses requires larger volumes or more concentrated suspensions, and often also the use of larger needles is necessary which is painful for the patient.

Several drugs are currently or have been on the market in sustained release formulations. A gonadotropin-releasing hormone agonist for the treatment of prostate cancer is marketed under the trade name Lupron Depot®, and human growth hormone for the treatment of growth hormone deficiency has been marketed under the trade name Nutropin Depot®.

An EPO analogue with reduced hepatic clearance in a sustained release formulation is disclosed in WO 01/30320. The EPO analogue has five changes in the amino acid sequence which effects a more extensive glycosylation.

### SUMMARY OF THE INVENTION

A sustained release formulation of a long-acting molecule will alleviate the problem of a very high drug load which is present in sustained release formulations of molecules with a rapid clearance.

Accordingly, the invention relates to a sustained release formulation comprising a protein modified so as to provide a reduced clearance, wherein said protein does not comprise a methionine in which the side chain sulphur has been modified.

In one embodiment, the invention relates to therapeutic methods comprising the administration of a therapeutically effective amount of a formulation according to the present invention.

In one embodiment, the invention provides a method for preparing a sustained release formulation comprising a protein modified so as to provide a reduced clearance, wherein said protein does not comprise a methionine in which the side chain sulfur has been modified, the method comprising the steps of
i) obtaining a protein, either by protein synthesis or by fermenting a suitable micro organism;
ii) modifying said protein ex vivo so as to obtain a reduced clearance; and
iii) formulating said modified protein in a sustained release formulation.

### DEFINITIONS

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

The term protein is intended to indicate two or more amino acid residues, which may be natural or unnatural, bonded by peptide bonds. The term includes proteins comprising further groups, such as e.g. prosthetic groups.

### DESCRIPTION OF THE INVENTION

In one embodiment, the invention relates to a sustained release formulation comprising a protein modified so as to provide a reduced clearance, wherein said protein does not comprise a methionine in which the side chain sulphur has been modified.

In one embodiment, said protein is a growth hormone compound.

In one embodiment, a growth hormone compound is intended to indicate human growth hormone.

In one embodiment, a growth hormone compound is intended to indicate human growth hormone which has been extended with a methionine at the N-terminal.

In one embodiment, growth hormone compound is intended to indicate a human growth hormone variant. In this context, a human growth hormone variant is intended to indicate proteins which exhibits at least 20% of the activity of human growth hormone, and wherein said protein exhibits at least 70% identity with human growth hormone. In particular, said protein exhibits at least 40%, such as at least 50%, such as at least 70%, such as at least 80%, such as at least 90%, such as least 95% of the activity of human growth, in combination with at least 80%, such as at least 90%, such as at least 95%, such as at least 97% identity with human growth hormone. Growth hormone activity may be measured as described in assay II herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

Particular examples of growth hormone compounds include human growth hormone, wherein amino acid No 172, 174, 176 and 178 as a group are replaced by one of the following groups of amino acids (R, S, F, R); (R, A, Y, R), (K, T, Y, K); (R, S, Y, R); (K, A, Y, R); (R, F, F, R); (K, Q, Y, R); (R, T, Y, H); (Q, R, Y, R); (K, K, Y, K); (R, S, F, S) or (K, S, N, R) as disclosed in WO 92/09690 (Genentech), which is incorporated herein by reference. Other examples of growth hormone compounds include human growth hormone with the following substitutions G120R, G120K, G120Y, G120F and G120E, as disclosed in U.S. Pat. No. 6,004,931 (Genentech), which is incorporated herein by reference.

Other examples of growth hormone compounds include human growth hormone with the following set of substitutions R167N, D171S, E174S, F176Y and I179T; R176E, D171S, E174S and F176Y; F10A, M14W, H18D and H21N; F10A, M14W, H18D, H21N, R167N, D171S, E174S, F176Y, I179T; F10A, M14W, H18D, H21N, R167N, D171A, E174S, F176Y, I179T; F10H, M14G, H18N and H21N; F10A, M14W, H18D, H21N, R167N, D171A, T175T and I179T; and F10I, M14Q, H18E, R167N, D171S and I179T, as disclosed in U.S. Pat. No. 6,143,523 (Genentech), which is incorporated herein by reference.

Other examples of growth hormone compounds include human growth hormone with the following set of substitutions H18A, Q22A, F25A, D26A, Q29A, E65A, K168A, E174A and G120K as disclosed in U.S. Pat. No. 6,136,536 (Genentech), which is incorporated herein by reference.

Other examples of growth hormone compounds include human growth hormone with the following set of substitutions H18D, H21N, R167N, K168A, D171S, K172R, E174S, I179T and wherein G120 is further substituted with either R, K, W, Y, F or E, as disclosed in U.S. Pat. No. 6,057,292 (Genentech), which is incorporated herein by reference.

Other examples of growth hormone compounds include human growth hormone with the following set of substitutions H18D, H21N, R167N, K168A, D171S, K172R, E174S and I179T, as disclosed in U.S. Pat. No. 5,849,535 (Genentech), which is incorporated herein by reference.

Other examples of growth hormone compounds include human growth hormone with the following set of substitutions H18D, H21D, R167N, K168A, D171S, K172R, E174S and I179T; and H18A, Q22A, F25A, D26A, Q29A, E65A, K168A and E174A, as disclosed in WO 97/11178 (Genentech), which is incorporated herein by reference.

Other examples of growth hormone compounds include human growth hormone with the following set of substitutions K168A and E174A; R178N and I179M; K172A and F176A; and H54F, S56E, L58I, E62S, D63N and Q66E as disclosed in WO 90/04788 (Genentech), which is incorporated herein by reference.

The modification of growth hormone compounds to obtain reduced clearance is typically done by covalently attach to the growth hormone compound a moiety which effects a decrease in the clearance rate. Various approaches can be used to decrease or prevent clearance. The moiety attached may increase the molecular size so as to decrease or prevent renal clearance; the moiety may shield the growth hormone compound from plasma proteases so as to prevent plasma protein induced break down; the moiety may bind to plasma proteins, such as e.g. albumin; and/or the moiety may shield receptor binding sites so as to prevent or decrease receptor induced clearance. It is to be understood that a given compound may be cleared from the body by a combination of mechanisms, and that the moiety attached may have an influence on more than one mechanism.

Typically, the moiety attached is polyethylene glycol (PEG) or a polymer derived from PEG (PEG and polymers derived from PEG will be referred commonly to as PEG), fatty acids, another protein, such as e.g. albumin, or a moiety which binds to plasma proteins, such as e.g. albumin. The moiety may be attached directly to the growth hormone compound, or it may be attached via a linker. Conveniently, cysteines, amines (N-terminal amino group or ε-amino in lysines) or other reactive groups present or introduced into growth hormone can be used as a points of attachment.

Growth hormone compounds is intended not to indicate compounds comprising methionine in which the sulfur in the side chain of said methionine has been modified. This is to say, that said side chain sulfur has the form C—S—C, and not e.g. C—S(O)₂—C, as in oxidized methionine.

It is known how to attach moieties to growth hormone compounds, and examples of relevant disclosures are given below.

U.S. Pat. No. 4,179,337 discloses methods or PEGylating growth hormone.

EP 458064, WO 95/11987 and WO 00/42175 all disclose PEGylation of Cys in growth hormone, where said cysteine may be natural or introduced into the sequence, e.g. by means of gene technology.

WO 03/044056 discloses a wide range of technologies which may be used to attached PEG to growth hormone.

Clark et al in J. Biol. Chem., 271, 21969-21977, 1996 also discloses methods of producing growth hormones with attached PEG.

Other documents, such as U.S. Pat. No. 5,045,312, WO 97/24445 and WO 01/79271 disclose that growth hormones with prolonged plasma residence time may be produced by fusing growth hormone to another protein, such as e.g. albumin.

Sustained release formulations are formulations designed to release the drug at a desired rate to give rise to an increased residence time. It is known that controlled release or administration of a drug may be obtained e.g. by implantable matrix devices, pumps, which may be implanted, gels or hydrogels, liposomes or micelles, crystalls, microspheres and reservoir devices. Microspheres and hydrogels are particular relevant for the present invention.

Microspheres are small (nm-μm) polymeric particles wherein a drug is encapsulated. These microspheres are typically injected subcutaneously or intramuscularly. There are three primary mechanisms by which drugs can be released from such release system: Diffusion, degradation, and swelling followed by diffusion. Any or all of these mechanisms may occur in a given release system.

Examples of suited polymers for microspheres include poly(D,L-lactide-co-glycolide) (PLGA), poly(carboxyphenoxypropane-co-sebaic acid) (p(CPP:SA)), poly(fatty acid dimmer-co-sebais acid, poly(trimellitylimidol-1-tyrosine-co-sebaic acid-co-1,3-bis(carboxyphenoxy) propane), polyorthoesters, polyanhydrides, polyamides, polyalkylcyanoacrylates and polyphosphazenes, poly(methacrylic acid), and triblock copolymers of PLG and PEG.

The drug release rate and profile depend on the properties of the microsphere and the interaction between the microsphere and the drug. Hydrophilic polymers (such as e.g. PGLA) allow water absorption into the body of the microspheres which results in bulk erosion. Bulk erosion is typically characterised by a bi- or even tri-phasic release profile; an initial burst where drug located near the surface is released; a second phase where the drug diffuses through water-filled pores and finally a third phase due to the final erosion and collapse of the polymer. More hydrophobic polymers (such as e.g. p(CPP:SA)) are eroded from the surface, which give rise to a more constant release rate. As the erosion proceeds, however, the surface area decreases, and the release rate will consequently decrease, too. It is to be understood that most microspheres are eroded by a combination of the two mechanisms, one of which may, nevertheless, dominate.

The nature of the polymer, of course, have a great impact on the release rate. Often, a biodegradable polymer is used, which mean that the hydrolytic rate of the polymer determines the release rate of the drug. More labile polymers will give rise to faster release rates. If co-polymers are used, a greater ratio of a labile monomer will also result in a faster release rate.

Specific interactions between the polymer and the drug may also delay the drug release. Clearly, the size of the microsphere will affect the rate of the drug release. As the size of the microspheres decrease, the surface area-to-volume ratio increases and so will the release rate. Other factors, such as changes of the pH in the microsphere due to break down products may also affect the break down rate to complicate prediction of the release rate and profile.

Various method to produce microspheres are known. Interfacial polymerisation, in general terms, employs a mixture of monomer(s) and an initiator, where the monomer(s) are polymerised in such a way that the growing polymer forms particles. Three common methods are known, namely suspension, emulsion and dispersion polymerisation. In suspension polymerisation, the monomer(s) and initiator is dissolved in a solvent, and the mixture is added to a suspension medium in which neither the monomer, initiator, solvent or resulting polymer are soluble. Droplets of the solvent is formed, e.g. by stirring, and as the polymerisation takes place, the polymers (microsphere) takes the form of the droplet.

Emulsion polymerisation is similar to suspension polymerisation, except that the initiator in soluble in the dispersion medium rather that in the monomer solvent.

Dispersion polymerisation is simpler in that it only employs a single phase. Monomer, initiator and a polymeric stabiliser are dissolved in a solvent, and as the polymer grows it precipitates and aggregates to form microspheres which are stabilised by adsorption of the stabiliser.

A very common method to produce microspheres is emulsion-solvent extraction/evaporation where preformed polymer is used. The polymer dissolved in a solvent is emulsified into a continuous phase which contains a stabiliser. Following the emulsification, the solvent is extracted into the continuous phase, which causes the polymer to harden to from droplets. In particular, water soluble drugs may be formulated in microspheres using a double emulsion process in which the an aqueous solution of the drug is first emulsified into the polymer containing solvent, and this water in oil emulsion is then emulsified into the continuous phase as described above.

Several extrusion methods are also known, wherein the microsphere components are forced through nozzles into an appropriate medium.

Hydrogels is another principle used for sustained release formulations. Hydrogels are three-dimensional polymer networks, composed of hydrophilic polymers, that swell, but do not dissolve in water. This network attains physical integrity and is made insoluble due to the presence of chemical and/or physical crosslinks.

Hydrogels that are capable of responding to the surrounding environment are termed physiologically responsive hydrogels. Some of the stimuli these hydrogels can respond to are changes in temperature, ionic strength and pH.

Some of the most common monomers used to form hydrogels with chemical crosslinks are 2-hydroxyethyl methacrylate, ethylene glycol dimethacrylate, N-isopropyl acrylamide, acrylic acid and methacrylic acid.

Examples of natural polymers from which hydrogels can be prepared are alginic acid, carrageenan, chitosan, polylysine, fibrin, collagen and gelatine.

Most of the above discussion of factors influencing the release rate and profile for microspheres is also relevant for hydrogels.

An attractive approach to drug delivery is to form a polymer matrix in situ from an injected aqueous polymer solution and to use the formed hydrogel as a depot for sustained release of the incorporated therapeutic drug, thus avoiding an invasive surgical placement. Any drug that is dissolved in the liquid precursor solution is then homogeneously dispersed in the polymer matrix and subsequently released over an extended period of time.

An example of in situ forming hydrogel polymer is poly(ethylene glycol)/poly( DL-lactic acid-co-glycolic acid) block copolymer.

General reference on sustained release formulations is made to Handbook of Pharmaceutical Controlled Release (Wise, D. L., ed. Marcel Dekker, New York, 2000); Drug and the Pharmaceutical Sciences vol. 99: Protein Composition and Delivery (MacNally, E. J., ed. Marcel Dekker, New York, 2000); and Varde et al in Expert Opin. Biol. Ther., 4, 35-51, 2004, which are all incorporated herein by reference.

The term “residence time” is used in its normal meaning, i.e., the time in which a compound is still present in the body/target organ. The residence time is conveniently determined from the time in which said compound still exerts a therapeutically relevant activity.

To determine if a modified growth hormone compound has an increased residence time, i.e. a reduced clearance, compared to the corresponding un-modified growth hormone compound, the following experiment is carried out. The two compounds in a suitable buffer is injected into suitable animals, such as e.g. mice, rats or humans. Blood samples are collected over time and analysed for the IGF-1 levels, as described in Assay I herein. The time at which the IGF-1 level falls under the therapeutic level is termed T. If T_{modified} has a value which is more than 2×, such as more than 3×, such as more than 10×, such as more than 100× the value of T_{un-modified}, then the modified growth hormone compound is said to have an increased residence time. If T_{modfied} has a value which is more than 2 hours, such as more than 4 hours, such as more than 12 hours, such as more than 24 hours, such as more than 3 days the value of T_{un-modified}, then the modified growth hormone compound is said to have an increased residence time.

To determine if a formulation gives rise to a sustained release, the following experiment is carried out. A growth hormone compound (modified or un-modified) in a buffer and in the formulation to be tested is injected into suitable animals, such as e.g. mice, rats or humans. Blood samples are collected over time and analysed for the IGF-1 levels, as described in Assay I herein. The time at which the IGF-1 level falls under the therapeutic level is termed T. If Tₜₑₛₜ has a value which is more than 2×, such as more than 3×, such as more than 10×, such as more than 100× the value of T_{buffer}, then the test formulation is said to be a sustained release formulation. If T_{modified} has a value which is more than 2 hours, such as more than 4 hours, such as more than 12 hours, such as more than 24 hours, such as more than 3 days the value of T_{un-modified}, then the tets formulation is said to be a sustained release formulation.

As discussed above, a sustained release formulation of a protein modified so as to give rise to a reduced clearance will alleviate problems associated with the similar formulation of the corresponding un-modified protein. In particular, the high clearance of the unmodified protein dictates a high drug load in the formulation. The high load requires that highly concentrated formulations are made, which is not always possible. In the situations where it is not possible to make formulations with sufficiently high drug loads, larger volumes have to be injected or the injections have to take place more frequently. A therapeutic regime involving the administration of compositions of the present invention will therefore comprise fewer injections with the corresponding convenience to the patient and increased compliance.

Furthermore, the high load will give rise to burst effects, i.e. a large release of drug immediately after the injection. It is known that certain adverse effects is related to such bursts. As an example, tunnel vision and oedema are related to peak values of growth hormone, and these adverse effects can be diminished if burst effects is reduced. Formulations of the present invention have a lower drug load and will therefore give rise to reduced burst affects.

An even greater reduction of the bursts and thus of the adverse effects can be achieved if the modified molecule interacts with the formulation matrix. Examples of such interactions include non-covalent interactions, such as ionic, van der Waals, hydrophobic and hydrogen bond interactions. Such interactions can easily be estimated by simple partition measurements.

In one embodiment, the invention relates to a sustained release formulation comprising a molecule that has been modified with a moiety so as to provide a reduced clearance, wherein said formulation and said moiety interact. In particular, said interaction is positive, i.e. there is a (non-covalent) binding between the moiety and the formulation.

In one embodiment, the molecule is a growth hormone compound conjugated to PEG, and the sustained release formulation comprises a hydrophobic polymer, such as PEG, PGLA, poly/methacrylic acid or co-polymers of the monomers constituting the aforementioned polymers e.g triblock co-polymers such as PLGA-PEG-PLGA.

In one embodiment, the decrease in clearance for the formulation of the present invention is higher than the sum of the decrease obtained from the modification of the protein and the decrease obtained from the sustained formulation.

In one embodiment, the invention relates to methods of preparing the formulations of the present invention. The unmodified protein may be obtained by any method known in the art. Relatively small proteins may be synthesised using standard protein synthetic methods. The unmodified protein may also be obtained from a fermentation of a suitable micro-organism which expresses the protein. The micro-organism may express the protein naturally, or it may have been genetically modified so as to express the protein. The protein may subsequently be isolated and purified by known methods.

The protein modification takes place ex vivo, and typically takes the form of attaching an organic moiety, as discussed above.

In one embodiment, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment inchildren, the method comprising administering to a patient in need thereof an effective amount of a composition according to the present invention

In one embodiment, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a composition according to the present invention.

In another embodiment, the invention relates to the use of a sustained release formulation comprising a growth hormone compound modified so as to provide a reduced clearance in the manufacture of a drug for the treatment of one of the above mentioned diseases.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law)

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

The effect of the present invention can be shown in a five-armed experiment. The following five formulations are injected into a suitable animal, such as mice, rats or humans.
1. Placebo
2. Un-modified growth hormone compound in buffer
3. Un-modified growth hormone compound in the sustained release formulation
4. Modified growth hormone compound in buffer
5. Modified growth hormone compound in the sustained release formulation
   Blood samples are drawn over a suitable period of time, and the samples are analysed for the IGF-1 level.

### PHARMACOLOGICAL METHODS

Assay (I) IGF-1 ELISA Assay

IGF-1 in rat or mouse plasma or serum is determined in a two-site immunoenzymometric assay in an OCTEIA™ kit obtainable from IDS Ltd., Boldon, England.

The samples are treated so as to inactivate the binding protein, IGF-BP 1-6. In the OCTEIA kit, a purified monoclonal anti-rat IGF-I is coated onto the inner surface of microtitre wells. The treated, diluted samples are incubated together with biotinylated polyclonal rabbit anti-rat IGF-I in the wells for two hours. The wells are then washed and horseradish peroxidase labelled avidin is added. After a further wash, a chromogenic compound, tetramethyl-benzidine, is added to develop colour. The colour of the stopped reaction is read in a microtitre plate reader, where the colour intensity is directly proportional to the amount of rat or mouse IGF-I present in the ample.

A similar assay with minor modifications can be used to determine human IGF-I.

Assay (I) BAF-3GHR Assay to Determine Growth Hormone Activity

The BAF-3 cells (a murine pro-B lymphoid cell line derived from the bone marrow) was originally IL-3 dependent for growth and survival. II-3 activates JAK-2 and STAT which are the same mediators GH is activating upon stimulation. After transfection of the human growth hormone receptor the cell line was turn into a growth hormone-dependent cell line. This clone can be used to evaluate the effect of different growth hormone samples on the survival of the BAF-3GHR.

The BAF-3GHR cells are grown in starvation medium (culture medium without growth hormoen) for 24 hours at 37° C., 5% CO₂.

The cells are washed and re-suspended in starvation medium and seeded in plates. 10 μl of growth hormone compound or human growth hormone in different concentrations or control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally the metabolic activity of the cells is measure in a fluorescence plate reader. The absorbance in the samples is expressed in % of cells not stimulated with growth hormone compound or control and from the concentration-response curves the activity (amount of a compound that stimulates the cells with 50%) can be calculated.

1. A sustained release formulation comprising a protein modified so as to provide a reduced clearance, wherein said protein does not comprise a methionine in which the side chain sulphur has been modified.

2. The formulation according to claim 1, wherein said protein is a growth hormone compound.

3. The formulation according to claim 2, wherein said growth hormone compound is human growth hormone

4. The formulation according to claim 1, wherein said formulation is a microsphere or a hydrogel.

5. A sustained release formulation comprising a protein modified with a moiety so as to provide a reduced clearance, wherein said formulation and said moiety interact positively.

6. The formulation according to claim 1, wherein the protein is a growth hormone compound conjugated to PEG, and the sustained release formulation comprises a hydrophobic polymer.

7. A method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment in children, the method comprising administering to a patient in need thereof an effective amount of a composition according to claim 2.

8. A method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a composition according to claim 2.

9. A method for the manufacture of a formulation according to claim 1, the method comprising the steps of
i) obtaining a protein, either by protein synthesis or by fermenting a suitable micro organism;
ii) modifying said protein ex vivo so as to obtain a reduced clearance; and
iii) formulating said modified protein in a sustained release formulation.
